(19) United States
(12) Patent Application Publication
(10) Pub. No.: US 2008/0146790 A1
(43) Pub. Date: Jun. 19, 2008

(54) Additive, Method, and Article For Dna Collection, Stabilization, and Purification

(76) Inventors: Daniel Grolz, Solingen (DE); Christian Lenz, Dusseldorf (DE); Uwe Oelmuller, Erkrath (DE); Manfred Weiler, Geneve (CH); Timothy A. Stevens, Warwick, NY (US)

Correspondence Address: David W. Highet, VP & Chief IP Counsel;Becton, Dickinson and Company, 1 Becton Drive, MC 110, Franklin Lakes, NJ, 07417-1880, US

(21) Appl. No.: 11/573,670

(22) PCT Filed: Aug. 16, 2005
(86) PCT No.: PCT/US2005/029059

§ 371 Date: Nov. 13, 2007

Related U.S. Application Data
(60) Provisional application No. 60/602,353, filed on Aug. 18, 2004.

(51) Int. Cl. C07H 1/08; C12M 1/33

(52) U.S. Cl. 536/25.41; 435/306.1

(57) ABSTRACT

A system for collection, stabilization, and preparing total DNA for direct purification from biological samples is provided.

### FIELD OF THE INVENTION

The present invention relates to collection, stabilization, and purification of total DNA from biological samples (such as whole blood) for subsequent diagnostic testing.

### BACKGROUND OF THE INVENTION

In recent years, there has been a significant interest in the field of biological, medical and pharmacological science in the study of nucleic acids obtained from biological samples. In particular, total DNA isolated from human whole blood, buccal cells and saliva can provide extensive information on the genetic origin and function of cells. This information may be used in clinical practice, e.g. in predisposition testing, HLA typing, identity testing, analysis of hereditary diseases and oncology. Moreover, total DNA of high quality is needed for many molecular diagnostic procedures—e.g. micro array analysis, quantitative or qualitative PCR, real time PCR etc.

Numerous strategies and methods have been described in the literature and in patents for biological sample collection or stabilization of nucleic acids in biological samples like whole blood, buccal cells, saliva and the like (for example U.S. Pat. No. 6,617,170). —In addition different strategies and methods exist to extract total DNA from biological samples, stabilized or not.

However, there is currently no blood collection system or method which meets all requirements for a total DNA process. Typically, various anticoagulants are used either alone or in conjunction with cell sustaining solutions to preserve a blood sample in an uncoagulated state. Some common anticoagulants are EDTA, sodium heparin and sodium citrate. In particular, sodium citrate solutions have been used for many years as anticoagulants and have been recommended for preserving yields of undegraded DNA during incubation at room temperature. [Gustafson et al., Anal. Biochem, 165 (2) (1987) 294-9].

Such blood collection systems may, however, generate micro clots after blood draw. This can lead to DNA contaminated with impurities in certain DNA isolation procedures.

Another disadvantage of current blood collection methods is that DNA undergoes degradation when stored at room temperature. Accordingly, for storage longer than 7 days the blood typically is frozen at −20° C. Even at a temperature in the range of −20° C. the DNA slowly degrades. Thus, for long term storage, it is generally necessary to freeze the blood at −70° C.

Methods for stabilization of total DNA are known. For example, in U.S. Pat. No. 6,458,546 a preservative solution for preserving a nucleic acid in a fluid such as a bodily fluid is disclosed. The solution contains a divalent metal chelator selected from ethylenediamine tetraacetic acid (EDTA), [ethylenebis(oxyethylenenitrilo)]tetraacetic acid (EGTA) and 1,2-bis(2-aminophenoxy)ethane-N,N,N′,N′-tetraacetic acid (BAPTA), and a chelator enhancing component.

There appears to be no teaching in this patent with regard to the isolation of the nucleic acids from such stabilized solutions. In fact direct isolation of the total DNA from blood samples mixed with this preservation solution does not seem to be possible. Instead, it appears that the stabilized samples have to be treated and incubated with additional solutions to further purify the DNA.

In United States Patent Applications 2003/0143566 and 2004/0009496, blood collection tubes are disclosed which contain a nucleic acid stabilization solution. In order to isolate total DNA from a blood collection tube containing a stabilization solution according to one of the above cited U.S. Patent Applications, it is believed that multiple steps and additional work is required.

One of the earliest methods for isolating nucleic acids is disclosed by Boom et al. (U.S. Pat. No. 5,234,809). Boom et al. describe a process for isolating nucleic acid from a nucleic acid-containing starting material, characterized by mixing the starting material with a chaotropic substance and a nucleic acid binding solid phase, separating the solid phase with the nucleic acid bound thereto from the liquid, washing the solid phase-nucleic acid complexes, and, if required, eluting the nucleic acid from the complexes. This patent focuses on nucleic acid purification only.

Therefore, there is a need for a system which comprises sample collection, total DNA stabilization and isolation which is easy to perform, rapid, cost effective and can be automated.

### SUMMARY OF THE INVENTION

In one aspect, the invention relates to a system having a container with an open top, a bottom, and a side wall extending therebetween defining a chamber. An additive is disposed in the chamber, the additive having at least one chaotropic substance, at least one detergent, and at least one buffer.

An aspect of the invention is a system wherein the biological sample is collected, the cells then are directly—without further manipulation steps—lysed and the total DNA is stabilized in a solution that allows directly the purification of the total DNA.

An aspect of the invention is to prepare a biological sample that is stable at room temperature for extended periods of time with little or no occurrence of DNA degradation.

A further aspect of the invention is to provide a system for stabilizing a biological sample, and particularly whole blood, immediately upon collection from the patient to inhibit or prevent degradation of total DNA when the sample is stored at room temperature.

Another aspect of the invention is to provide a system of collecting and stabilizing a blood sample in a container having an additive. The additive contains a chaotropic salt, e.g., a guanidinium salt, for example guanidinium hydrochloride, guanidinium thiocyanate, aminoguanidinium hydrochloride, N,N′-diamino-guanidinium hydrochloride, or 1-methylguanidinium hydrochloride, and/or a lithium salt, i.e. lithium chloride, lithium bromide, lithium nitrate, and/or sodium iodide, and/or urea; a detergent, for example Triton®, particularly Triton® X-100 (octylphenoxypolyethoxyethanol) and/or SDS and/or TWEEN (Polyoxyethylenemonosorbate); and a buffer, particularly TRIS, e.g., at a pH 6-8. The additive optionally contains chelating agents, including, but not limited to, ethylenediamine tetraacetic acid (EDTA) and/or ethylene glycol-bis(2-aminoethylether)-N,N,N′,N′-tetraacetic acid (EGTA).

A further aspect of the invention is to provide an additive which lyses cells in the collection device, inactivates DNases, stabilizes total DNA and adjusts conditions for subsequent total DNA isolation.

Another aspect of the invention is to provide a collection container such as an evacuated collection tube, for receiving and collecting a biological sample where the container is pre-filled with a measured quantity of such an additive.

### BRIEF DESCRIPTION OF THE DRAWINGS

The following is a brief description of the drawings, in which:

FIG. 1 is a cross-sectional side view of the container in one embodiment of the invention;

FIG. 2 is an agarose gel showing the purity and size of the total DNA isolated according to Example 1;

FIG. 3 is an agarose gel showing the purity and size of the total DNA isolated according to Example 2;

FIG. 4 is an agarose gel showing the purity and size of the total DNA isolated according to Example 3;

FIG. 5 is an agarose gel showing the purity and size of the total DNA isolated according to Example 4;

FIG. 6 is an agarose gel showing the purity and size of the total DNA isolated according to Example 5;

FIG. 7 is an agarose gel showing the purity and size of the total DNA isolated according to Example 6.

FIG. 8 is an agarose gel showing the purity and size of the total DNA isolated according to Example 7.

FIG. 9 is an agarose gel showing the purity and size of the total DNA isolated according to Example 8.

FIG. 10 is an agarose gel showing the purity and size of the total DNA isolated according to Example 9.

### DETAILED DESCRIPTION OF THE INVENTION

In one aspect, the invention relates to a system having a container with an open top, a bottom, and a side wall extending therebetween defining a chamber. An additive is disposed in the chamber, the additive having at least one chaotropic substance, at least one detergent and at least one buffer.

The additive is directed to acting on nucleic-acid containing biological samples, to stabilize the nucleic acid and prepare it for subsequent processing. Examples of biological samples include, but are not limited to, cell-containing compositions such as red blood cell concentrates, platelet concentrates, leukocyte concentrates, tumor cells, bone marrow, aspirates, tissue, fine needle aspirates and cervical samples; body fluids such as whole blood, plasma, serum, urine, cerebral spinal fluid, and sputum; bacteria or eucaryotic microorganisms; or tissues, body swabs and body smears.

In a particular embodiment, the biological fluid is whole blood.

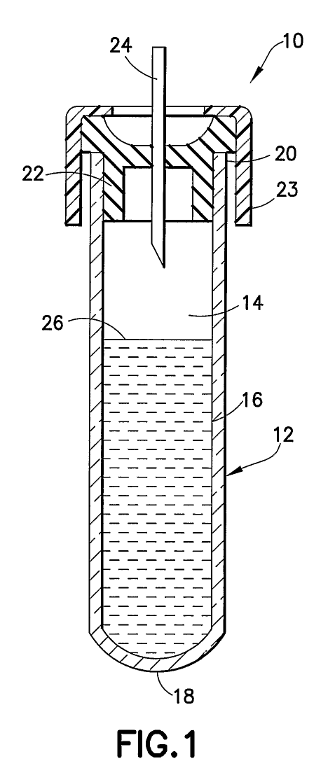

In one embodiment, reflected in FIG. 1, the system includes a device 10 for drawing a blood sample directly from an animal, and particularly a human patient for stabilizing the nucleic acids and blocking DNA degradation immediately at the point of collection. Device 10 constitutes a container 12 defining a chamber 14. In the embodiment illustrated, container 12 is a hollow tube having a side wall 16, a closed bottom end 18 and an open top end 20. Container 12 is dimensioned for collecting a suitable volume of a biological fluid sample. A resilient closure 22 is positioned in open top end 20 to close container 12. Preferably, closure 22 forms a seal capable of effectively closing container 12 and retaining a biological sample in chamber 14, as well as retaining a reduced pressure environment. A protective shield 23 overlies closure 22.

Container 12 can be made of glass, plastic or other suitable materials. Plastic materials can be oxygen impermeable materials or contain an oxygen impermeable layer. Alternatively, container 12 can be made of a water and air permeable plastic material. Preferably, chamber 14 maintains a pressure differential between atmospheric pressure and is at a pressure less than atmospheric pressure. The pressure in chamber 14 is selected to draw a predetermined volume of a biological sample into chamber 14. Typically, a biological sample is drawn into chamber 14 by piercing closure 22 with a needle 24 or cannula as known in the art. An example of a suitable container 12 and closure 22 are disclosed in U.S. Pat. No. 5,860,937 to Cohen. Examples of suitable thermoplastic materials include polycarbonates, polyethylene, polypropylene, polyethylene terephthalate. Container 12 has a suitable dimension selected according to the required volume of the biological sample being collected. In one embodiment, container 12 has a tubular shape with an axial length of about 75 to 100 mm and a diameter of about 13 mm to 16 mm. Suitable materials for closure 22 include, for example, silicone rubber, natural rubber, styrene butadiene rubber, ethylene-propylene copolymers and polychloropropene.

For collection of swabs or other samples, a container can be provided without a vacuum and can in such a case utilize a conventional cap, such as a screw cap. Similarly, a syringe-type device can be used, such as sold by Sarstedt AG (Germany).

Container 12 also contains the additive 26 according to the present invention. (The term “additive” is used herein to refer to the stabilizing composition present in the system, e.g., in the container, of the invention.) The additive 26 can be in any suitable physical form, but is preferably aqueous. In one embodiment, the internal pressure of container 12 and the volume of the additive 26 are selected to provide the necessary concentration of the additive for the volume of the biological sample collected, to provide desired stabilization to enable direct purification of total DNA. In one preferred embodiment, the internal pressure of container 12 is selected to draw a predetermined volume of about 1 ml of a biological sample into container 12 containing an effective volume of the additive 26 for stabilizing the volume of the biological sample. In alternative embodiments, container 12 can have an internal pressure at substantially atmospheric pressure. Preferably, container 12 is pre-filled with the additive by the manufacturer and packaged in a ready to use form. Typically, the packaged container is sterile and packaged in sterile packaging materials.

Water loss by evaporation of the additive through the permeable wall of the container increases the concentration of the stabilizing agent and decreases the pressure within the container. The diffusion of oxygen through the wall of the tube has the effect of decreasing the vacuum in the container. The water and oxygen permeability properties of the container are selected to maintain the desired pressure differential within the container for the desired shelf life of the container. The shelf life is optimized by balancing the oxygen permeability with the water loss. The container advantageously has a shelf life of at least about one year, and preferably longer.

In addition to the container of FIG. 1, other sample collection containers and devices are also possible.

The additive 26 preferably contains at least one agent in a concentration effective for stabilizing nucleic acids in the biological sample, and particularly in a whole blood sample. Advantageously, the agent provides the stabilization function without detrimentally affecting the subsequent purification step. For example, the agent does not interfere with binding of the total DNA onto a nucleic acid binding solid phase.

Typically, the additive 26 is an aqueous solution of a mixture of one or more stabilizing agents. The stabilizing agents advantageously stabilize DNA and RNA including mRNA, tRNA, snRNA, lower molecular weight (LMW) RNA, rRNA and cRNA and are able to block or inhibit ex vivo nucleic acid degradation in a biological sample during storage at room temperature (15-25° C.). Examples of suitable agents for stabilizing and preserving nucleic acids and/or preventing gene induction include cationic compounds, detergents, chaotropic substances, and mixtures thereof. Advantageously, the additive comprises at least one chaotropic substance, at least one detergent, and at least one buffer.

Useful chaotropic substances include but are not limited to urea, sodium iodide, guanidinium salts and derivatives thereof (such as amino substituted and alkyl-substituted guanidinium salts), and lithium salts (advantageously salts of lithium and a mineral acid, such as a lithium halide, e.g. lithium chloride, lithium bromide or lithium nitrate). A useful concentration of urea is 3-7 M in the additive, a useful concentration of sodium iodide salt is 4-6 M in the additive, a useful concentration of guanidinium salt is 2-6 M in the additive, and a useful concentration of lithium salt is 1-5 M in the additive. The chaotropic compounds generally perform the function of denaturing proteins and thereby increasing the permeability of cell membranes.

The additive may contain mixtures of chaotropic substances—for example mixtures of lithium salts, or mixtures of guanidinium and lithium salts, or mixtures of urea and a lithium salt such as lithium chloride.

The detergents can be anionic detergents, cationic detergents or nonionic detergents. The anionic detergent can be, for example, sodium dodecyl sulfate (SDS). Nonionic detergents can be, for example, ethylene oxide condensation products, such as ethoxylated fatty acid esters of polyhydric alcohols. A preferred nonionic detergent is a polyoxyethylene sorbitan monolaurate sold under the trade name TWEEN® 20 by Sigma Chemical Co. Other TWEEN® detergents are also suitable. Other suitable detergents can be taken from the group of Triton® detergents (octylphenoxypolyethoxyethanol), advantageously Triton® X-100 (also available from Sigma). The detergents are included in an effective amount to lyse the cells, with ranges of 1.5-20% (v/v) typically being effective. The detergents may also form micelles and other complexes with the nucleic acids, thereby protecting DNA via other mechanisms.

The pH of the mixture of the biological sample and additive is typically controlled by the inclusion of a buffer. An advantageous pH range of the sample/additive mixture is pH 6 to pH 9. Suitable buffer substance are well known from the state of the art. TRIS (tris (hydroxymethyl)aminomethane) is a useful buffer. However, the pH can be adjusted with other suitable buffers known in the art, including but not limited to HEPES (N-(2-Hydroxyethyl)piperazine-N′-(2-ethane-sulfonic acid)) salts, MOPS (3-(N-Morpholino)propanesulfonic acid salts), MES ((2-Morpholino)ethanesulfonic acid salts), citrate buffers or phosphate buffers. Depending on the particular buffer, the appropriate concentration will vary. Typical concentrations fall in the range of 5-500 mM in the additive, more advantageously 10-100 mM.

The additive may also contain a nucleic acid binding solid phase, e.g., magnetic particles or other solid phases useful for binding to and separating nucleic acids, as known in the art.

Useful additives include, in an aqueous solution:

1-5 M Lithium salt—preferably lithium chloride, lithium bromide and/or lithium nitrate, 2.5-14% (v/v) Triton® X-100, and 10-100 mM (advantageously 50 mM) TRIS (pH 6-8). In a preferred embodiment one volume of the biological sample—preferably blood—is mixed with 1-4 volumes—most preferably 2.5-3 volumes—of this type of additive;

2 M-6 M Guanidinium salt—preferably guanidinium hydrochloride, guanidinium thiocyanate, amino-guanidinium hydrochloride, N,N′-diamino-guanidinium hydrochloride and/or 1-methyl-guanidinium hydrochloride, 1.5-14% (v/v) Triton® X-100, and 10-100 mM (advantageously 50 mM) TRIS (pH 6-9, preferably pH 6.5-8.5). In a preferred embodiment one volume of the biological sample—preferably blood—is mixed with 1-4 volumes—most preferably 2.5-3.0 volumes—of this type of additive;

3-7 M Urea, 5-14% (v/v) Triton® X-100, and 10-100 mM (advantageously 50 mM) TRIS (pH 6-8, preferably pH 8). In a preferred embodiment one volume of the biological sample—preferably blood—is mixed with 2.5-6 volumes—most preferably 3-4 volumes—of this type of additive;

3-7 M Urea, 1.5-20 (advantageously 2) % (v/v) SDS, and 10-100 mM (advantageously 50 mM) TRIS (pH 6-8, preferably pH 8). In a preferred embodiment one volume of the biological sample—preferably blood—is mixed with 2.5-6 volumes—most preferably 3-4 volumes—of this type of additive;

3-7 M Urea, 1-2 M lithium chloride, 5-14% (v/v) Triton® X-100, and 10-100 mM (advantageously 50 mM) TRIS (pH 6-8, preferably pH 8). In a preferred embodiment one volume of the biological sample—preferably blood—is mixed with 2-6—most preferably 3-4 volumes of this type of additive;

2 M-5 M Guanidinium salt—preferably guanidinium hydrochloride, guanidinium thiocyanate, amino-guanidinium hydrochloride, N,N′-diamino-guanidinium hydrochloride or 1-methyl-guanidinium hydrochloride—, 1-3 M lithium chloride, 5-14% (v/v) Triton® X-100, and 10-100 mM (advantageously 50 mM) TRIS (pH 6-8, preferably pH 6). In a preferred embodiment one volume of the biological sample—preferably blood—is mixed with 2-4—most preferably 2.5-3.5 volumes of this type of additive; and

4-6 M Sodium iodide, 5-14% Triton® X-100, and 10-100 mM (advantageously 50 mM) TRIS (pH 6-8, preferably pH 6). In a preferred embodiment one volume of the biological sample—preferably blood—mixed with 3-6 volumes—most preferably 3-4 volumes—of this type of additive.

In addition, the additives may contain one or more chelating agents, preferably EDTA, EGTA, and/or salts of EDTA and EGTA. EDTA or salts thereof are advantageously present in an amount of 10 to 100 mM in the additive. EGTA or salts thereof are advantageously present in an amount of 1 to 10 mM in the additive. The chelating agents function to remove bound metals which may be required for stability of cell membranes, thereby contributing to disintegration of cell membranes.

In one embodiment, the system of the invention is used by obtaining a biological sample and introducing the sample into a container containing an additive. Advantageously, the biological sample is immediately introduced into the collection container. More advantageously, the biological sample is withdrawn from the patient directly into the collection container without any intervening process or handling steps, such that the sample mixes with the additive immediately, to prevent or inhibit nucleic acid decomposition. It has been found that collecting the biological sample directly from the patient, such as when collecting a whole blood sample directly into a reduced-pressure tube containing the additive, substantially prevents or reduces the decomposition of the nucleic acids that otherwise occurs when the sample is stored before combining with the additive.

Total DNA purification is typically achieved by transferring the lysate from the collection device directly onto a nucleic acid binding solid phase, advantageously without prior mixing and/or incubation with additional reagents, buffers or enzymes. DNA is bound onto the nucleic acid binding solid phase, washed and eluated. All these steps are well known in the art, and are reflected in the Examples below. In the case where the container itself contains a solid phase, as shown in Example 10 such binding occurs in the container. Subsequent steps generally involve removal of lysate, followed by washing and elution of nucleic acid.

Therefore, the first steps of total DNA isolation are performed within the collection device. The sample collection, total DNA stabilization and subsequent total DNA isolation are chemically linked and optimized. The method for total DNA isolation is suitable for high throughput purification and can easily be automated.

### EXAMPLES

### Example 1

The buffers used in the examples have the following compositions:

| | |
|---|---|
| washing buffer 1: | 3M Guanidinium hydrochloride, 57% ethanol (EtOH) |
| washing buffer 2: | 10 mM TRIS-Cl pH 7.5, 100 mM NaCl, 70% EtOH |
| elution buffer: | 10 mM TRIS-Cl pH 9.0, 0.5 mM EDTA |

Venous whole blood from one donor was drawn into PAXgene Blood DNA tubes (PreAnalytiX®). Directly after draw 350 μl anticoagulated whole blood were mixed with 350 μl of the buffers listed in Table 1 and stored for 12 hours at room temperature.

TABLE 1

| Buffer | Composition |
|---|---|
| L1 | 3 M LiCl, 5% Triton ® X-100, 50 mM TRIS pH 6 |
| L2 | 3 M LiCl, 10% Triton ® X-100, 50 mM TRIS pH 6 |
| L3 | 4 M LiCl, 2.5% Triton ® X-100, 50 mM TRIS pH 6 |
| L4 | 4 M LiCl, 5% Triton ® X-100, 50 mM TRIS pH 6 |
| L5 | 4 M LiCl, 10% Triton ® X-100, 50 mM TRIS pH 6 |

For each additive, four samples were processed. Lysates (700 μl) were pipetted onto a 96 well QIAamp® plate (QIAGEN®) and processed with a manual vacuum chamber. After transfer of all samples, the lysates were sucked through the silica membrane by vacuum. Bound DNA was subsequently washed with 500 μl washing buffer 1 and 500 μl washing buffer 2 using vacuum. The plate was dried by centrifugation for 15 min at 6000 rpm. After drying, the total DNA was eluted from the membrane with 200 μl elution buffer by centrifugation for 4 min at 6000 rpm.

The eluted DNA was analyzed as follows:

Spectrophotometry, absorbance at 260 and 280 nm

Real time PCR on ABI Prism 7700: β actin assays (Applied Biosystems) using 2 μl eluate as template, 25 μl total reaction volume.

Agarose gel electrophoresis: 0.8% agarose in 1×TBE buffer, run 120 min at 100 V with 20 μl of eluate.

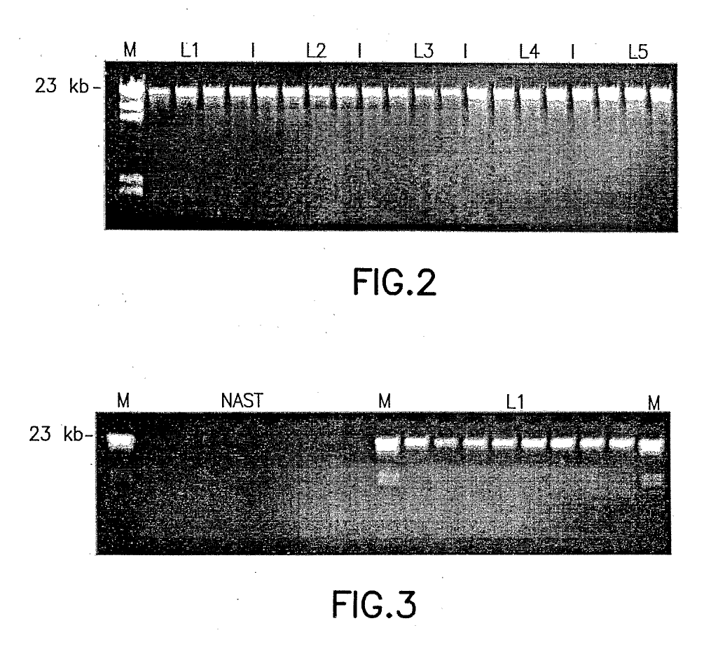

The results are shown in Table 2, with the gel results in FIG. 2.

TABLE 2

| Addi- | AV yield | SD | AV | SD | PCR | SD |
|---|---|---|---|---|---|---|
| tive | [μg] | yield | A260/A280 | A260/A280 | AV CT | CT |
| L1 | 2.06 | 0.23 | 1.92 | 0.09 | 23.0 | 0.70 |
| L2 | 3.24 | 0.20 | 2.02 | 0.05 | 22.5 | 0.25 |
| L3 | 2.77 | 0.39 | 1.89 | 0.30 | 23.2 | 0.24 |
| L4 | 3.45 | 0.25 | 2.04 | 0.00 | 22.4 | 0.31 |
| L5 | 3.68 | 0.28 | 1.90 | 0.22 | 22.3 | 0.43 |

As shown by Table 2 and FIG. 2 the purified DNA is of high purity, can be amplified in a real time TaqMan® assay and is visible as an intact band on an agarose gel. Moreover, DNA from whole blood can be purified with additive L1 to L5 by direct loading onto a silica membrane without further incubations or adjustments.

### Example 2

Venous whole blood from four donors was drawn into PAXgene Blood DNA tubes (PreAnalytiX®) and 3.5 ml NAST-Vacuette® Blood Sampling tubes (Antigene Biotech GmbH, Germany, Cat. No. 454282). From the PAXgene Blood DNA tubes, 1 ml of anticoagulated whole blood was mixed with 2 ml additive L1. The NAST-Vacuette already contained a nucleic acid stabilization solution. The lysates and NAST-Vacuettes were stored for 7 days at 25° C.

The composition of the additive L1 is shown in Table 3:

TABLE 3

| Additive | Composition |
|---|---|
| NAST | nucleic acid stabilization buffer |
| L1 | 5.6 M guanidinium hydrochloride, 10% Triton ® X-100, |
| | 50 mM TRIS pH 6 |

From each donor, two samples with additive L1 and two samples from the NAST-Vacuettes® were processed. L1 lysates (450 μl, equivalent to 150 μl blood) were pipetted onto a 96 well QIAamp® plate and processed as described in Example 1. For samples taken from the NAST-Vacuettes® 400 μl lysate was mixed with 400 μl of a pre-incubation solution (PRIS, Antigene Biotech GmbH, Cat. No. 07-2002) and incubated for 15 min at room temperature to remove crystals, as described in a standard operating procedure from Antigene Biotech GmbH. 800 μl lysate (equivalent to ˜150 μl blood) were pipetted onto the same 96 well QIAamp plate and processed in parallel with the L1 lysates with a manual vacuum chamber as described in Example 1. The eluates were analyzed as described in Example 1.

The results are shown in Table 4, with the gel results in FIG. 3.

TABLE 4

| Addi- | AV yield | SD | AV | SD | PCR | SD |
|---|---|---|---|---|---|---|
| tive | [μg] | yield | A260/A280 | A260/A280 | AV CT | CT |
| NAST | 0.57 | 0.19 | 3.15 | 3.61 | 29.9 | 0.70 |
| L1 | 1.84 | 0.42 | 1.93 | 0.05 | 22.1 | 0.69 |

As shown by Table 4 and FIG. 3, direct loading of lysates from the NAST-Vacuette® pre-incubated with PRIS-solution leads to low yield, low purity and high CT values in real time PCR. It can be seen that DNA from whole blood lysed with additive L1 can be purified without further incubations or adjustments. The DNA purified with additive L1 is of high purity, can be amplified in a real time TaqMan® assay and is visible as an intact band on an agarose gel. Moreover, DNA from whole blood mixed with buffer L1 is stable for at least 7 days at 25° C.

### Example 3

For each additive listed in Table 5, venous whole blood (1 ml) from one donor was drawn into a Vacutainer® blood collection tube (13 mm×75 mm, Becton, Dickinson & Company) without anticoagulant. Directly after blood collection the Vacutainer® was opened and 3 ml of additive was added. Blood and additive were mixed by inverting the tube 3 times.

The compositions of the additives are shown in Table 5:

TABLE 5

| Additive | Composition |
|---|---|
| L1 | 4.9 M guanidinium hydrochloride, 8.9% Triton ® X-100, |
| | 50 mM TRIS pH 6 |
| L2 | 4.5 M guanidinium hydrochloride, 8% Triton ® X-100, |
| | 50 mM TRIS pH 6 |
| L3 | 3.7 M guanidinium hydrochloride, 6.6% Triton ® X-100, |
| | 50 mM TRIS pH 6 |
| L4 | 5.6 M guanidinium hydrochloride, 6.5% Triton ® X-100, |
| | 50 mM TRIS pH 6 |
| L5 | 5.6 M guanidinium hydrochloride, 4% Triton ® X-100, |
| | 50 mM TRIS pH 6 |

The samples were stored in the primary blood collection tubes for 27 days at 25° C.

After storage the primary blood collection tubes were placed on a BioRobot® MDx. 600 μl of lysate were transferred into one well of a 96 well QIAamp® plate. After the complete transfer of all samples, the lysates were sucked through the silica membrane by vacuum. Bound DNA was subsequently washed with 520%1 washing buffer 1, 775 μl washing buffer 2 and 1020 μl ethanol. Each washing solution was applied with a dispenser and sucked through the membrane by vacuum. After drying the plate the total DNA was eluted from the membrane with 150 μl elution buffer.

The eluted DNA was analyzed as described in Example 1.

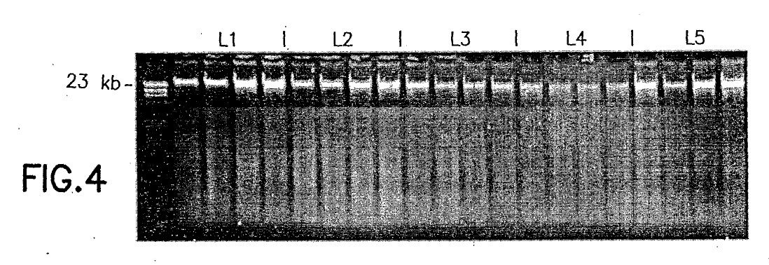

The results are shown in Table 6, with the gel shown in FIG. 4:

TABLE 6

| Addi- | AV yield | SD | AV | SD | PCR | SD |
|---|---|---|---|---|---|---|
| tive | [μg] | yield | A260/A280 | A260/A280 | AV CT | CT |
| L1 | 4.48 | 0.62 | 1.88 | 0.03 | 21.5 | 0.21 |
| L2 | 4.71 | 0.59 | 1.83 | 0.15 | 21.9 | 0.89 |
| L3 | 4.39 | 0.91 | 1.88 | 0.04 | 22.2 | 0.90 |
| L4 | 2.01 | 0.11 | 1.89 | 0.02 | 23.5 | 0.65 |
| L5 | 4.18 | 0.57 | 1.78 | 0.03 | 21.3 | 0.60 |

Core extraction time for 96 samples with additives L1 to L5 was 58 min.

As shown in Table 6 and FIG. 4, DNA from whole blood lysed with additive L1 to L5 can be purified without incubation or adjustments on the automated platform BioRobot® MDx. The purified DNA is of high purity, can be amplified in a real time TaqMan® assay and is visible as an intact band on an agarose gel. The DNA is stable in additives L1 to L5 for at least 28 days at 25° C.

### Example 4

### Material and Methods

Venous whole blood from one donor was drawn into Monovette® blood collection devices (Sarstedt, 9 ml draw volume) without anticoagulant. Directly after blood collection, 1 volume of blood taken from the devices was mixed with 3 volumes of each of additives L1-L8.

The compositions of the additives are shown in Table 7:

TABLE 7

| Additive | composition |
|---|---|
| L1 | 4.0 M guanidinium hydrochloride, 12% Triton ® X-100, |
| | 100 mM EDTA, 50 mM TRIS pH 7 |
| L2 | 4.0 M guanidinium hydrochloride, 12% Triton ® X-100, |
| | 80 mM EDTA, 50 mM TRIS pH 7 |
| L3 | 4.0 M guanidinium hydrochloride, 12% Triton ® X-100, |
| | 50 mM EDTA, 50 mM TRIS pH 7 |
| L4 | 4.0 M guanidinium hydrochloride, 12% Triton ® X-100, |
| | 40 mM EDTA, 50 mM TRIS pH 7 |
| L5 | 4.0 M guanidinium hydrochloride, 12% Triton ® X-100, |
| | 20 mM EDTA, 50 mM TRIS pH 7 |
| L6 | 4.0 M guanidinium hydrochloride, 12% Triton ® X-100, |
| | 10 mM EGTA, 50 mM TRIS pH 7 |
| L7 | 4.0 M guanidinium hydrochloride, 12% Triton ® X-100, |
| | 5 mM EGTA, 50 mM TRIS pH 7 |
| L8 | 4.0 M guanidinium hydrochloride, 12% Triton ® X-100, |
| | 50 mM TRIS pH 7 |

The lysates were aliquotted into 13 nm×75 mm tubes (Becton, Dickinson and Company), 8 replicates per lysis buffer (L1-L8) and stored for 36 days at room temperature.

After storage the tubes were placed on a BioRobot MDx. For total DNA extraction on the BioRobot MDx, 800 μl of lysate were transferred onto a well of a 96 well QIAprep plate (QIAGEN).

After the complete transfer of all samples, the lysates were sucked through the silica membrane by vacuum. Bound DNA was subsequently washed with 520 μl washing buffer 1, 775 μl washing buffer 2 and 1020 μl ethanol (EtOH). Each washing solution was applied with a dispenser and sucked through the membrane by vacuum. After drying the total DNA was eluted from the membrane with 200 μl water.

The eluted DNA was analyzed as described in Example 1.

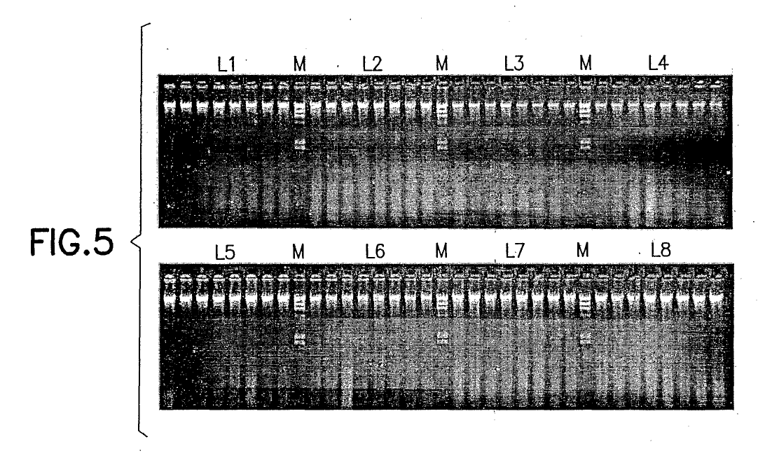

The results are shown in Table 8, with the gel shown in FIG. 5:

TABLE 8

| Addi- | AV yield | SD | AV | SD | PCR | SD |
|---|---|---|---|---|---|---|
| tive | [μg] | yield | A260/A280 | A260/A280 | AV CT | CT |
| L1 | 3.9 | 0.2 | 1.96 | 0.03 | 22.26 | 0.60 |
| L2 | 4.8 | 0.5 | 1.93 | 0.03 | 21.72 | 0.48 |
| L3 | 4.0 | 0.9 | 1.90 | 0.04 | 22.41 | 0.75 |
| L4 | 4.3 | 0.2 | 1.90 | 0.05 | 21.67 | 0.44 |
| L5 | 4.1 | 0.1 | 1.92 | 0.02 | 21.71 | 0.31 |
| L6 | 4.0 | 0.1 | 1.92 | 0.04 | 21.75 | 0.39 |
| L7 | 2.9 | 0.2 | 1.94 | 0.04 | 23.02 | 0.48 |
| L8 | 3.7 | 0.8 | 1.96 | 0.04 | 22.69 | 0.40 |

Core extraction time for 96 samples was 59 min.

As shown by Table 8 and FIG. 5, DNA from whole blood lysed with additives L1-L8 can be purified without protease incubation or adjustment of binding conditions on the automated platform BioRobot® MDx. The purified DNA is of high purity, can be amplified in a real time TaqMan® assay and is visible as an intact band on an agarose gel. The DNA is stable in additives L1-L8 for at least 36 days at room temperature.

### Example 5

Venous whole blood from one donor was drawn into Monovette® blood collection devices (Sarstedt, 9 ml draw volume) without anticoagulant. Directly after blood collection, 1 volume of blood taken from the devices was mixed with 2.8 volumes of each of additives L1-L4.

The compositions of the additives are shown in Table 9:

TABLE 9

| Additive | composition |
|---|---|
| L1 | 3.0 M guanidinium hydrochloride, 10% Triton ® X-100, |
| | 50 mM EDTA, 50 mM TRIS pH 7 |
| L2 | 3.5 M Guanidinium hydrochloride, 10% Triton ® X-100, |
| | 50 mM EDTA, 50 mM TRIS pH 7 |
| L3 | 4.0 M Guanidinium hydrochloride, 10% Triton ® X-100, |
| | 50 mM EDTA, 50 mM TRIS pH 7 |
| L4 | 4.5 M Guanidinium hydrochloride, 10% Triton ® X-100, |
| | 50 mM EDTA, 50 mM TRIS pH 7 |

The lysates were aliquotted into 13 mm×75 mm tubes (Becton, Dickinson and Company, 24 replicates per additive) and directly placed on a BioRobot MDx for processing. For total DNA extraction, 800 μl of lysate were transferred into a well of a 96 well QIAprep plate.

After the complete transfer of all samples, the lysates were sucked through the silica membrane by vacuum. Bound DNA was subsequently washed with 520 μl washing buffer 1, 775 μl washing buffer 2 and 1020 μl ethanol. Each washing solution was applied with a dispenser and sucked through the membrane by vacuum. After drying, the plate DNA was eluted from the membrane with 200 μl water.

The eluted DNA was analyzed as described in Example 1.

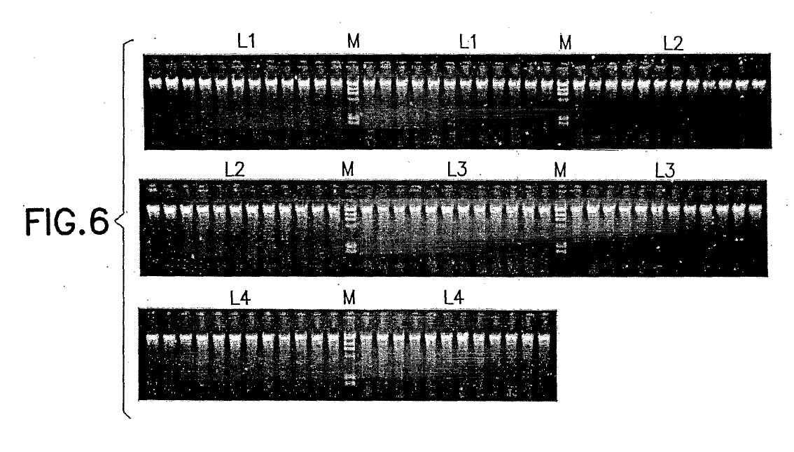

The results are shown in Table 10, with the gel shown in FIG. 6:

TABLE 10

| Addi- | AV yield | SD | AV | SD | PCR | SD |
|---|---|---|---|---|---|---|
| tive | [μg] | yield | A260/A280 | A260/A280 | AV CT | CT |
| L1 | 3.8 | 0.6 | 1.76 | 0.13 | 21.12 | 1.14 |
| L2 | 3.9 | 0.7 | 1.80 | 0.09 | 21.71 | 0.26 |
| L3 | 3.5 | 0.4 | 1.82 | 0.10 | 21.82 | 0.36 |
| L4 | 3.4 | 0.3 | 1.84 | 0.05 | 21.72 | 0.32 |

Core extraction time for 96 samples with additives L1 to L4 was 57 min.

As shown by Table 10 and FIG. 6, DNA from whole blood lysed with additives L1-L4 can be purified without protease incubation or adjustment of binding conditions on the automated platform BioRobot MDx. The purified DNA is of high purity, can be amplified in a real time TaqMan® assay and is visible as an intact band on an agarose gel.

### Example 6

Venous whole blood (1 ml) from one donor was drawn into a Vacutainer® blood collection tube (13 mm×75 mm) without anticoagulant. For each additive in Table 11, directly after blood collection the tube was opened and 3 ml additive was added. Blood and additive were mixed by inverting the tube 3 times. The lysate was stored at 25° C. The compositions of the additives are shown in Table 11:

TABLE 11

| Additive | Composition |
|---|---|
| L1 | 4.9 M 1-methyl-guanidinium hydrochloride, |
| | 8.9% Triton ® X-100, 50 mM TRIS pH 6 |
| L2 | 3.1 M amino-guanidinium hydrochloride, |
| | 6.7% Triton ® X-100, 25 mM TRIS pH 6 |
| L3 | 4.6 M amino-guanidinium hydrochloride, |
| | 10% Triton ® X-100, 25 mM TRIS pH 6 |
| L4 | 2 M N,N′diamino-guanidinium hydrochloride, |
| | 8.9% Triton ® X-100, 25 mM TRIS pH 6 |
| L5 | 1.7 M N,N′-diamino-guanidinium hydrochloride, |
| | 8.9% Triton ® X-100, 25 mM TRIS pH 6 |

The samples were stored in the primary blood collection tubes for 28 days at 25° C.

After storage, the primary blood collection tubes were placed on a BioRobot® MDx. Processing on BioRobot® MDx was performed with 600 μl lysate according to the procedure described in Example 3. The eluted DNA was analyzed as described in Example 1.

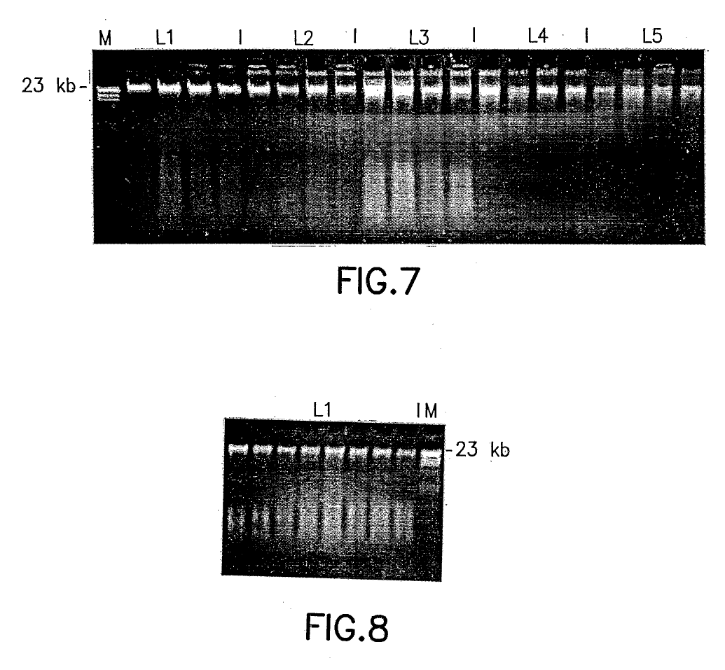

The results are shown in Table 12, with the gel shown in FIG. 7.

TABLE 12

| Addi- | AV yield | SD | AV | SD | PCR | SD |
|---|---|---|---|---|---|---|
| tive | [μg] | yield | A260/A280 | A260/A280 | AV CT | CT |
| L1 | 3.95 | 0.77 | 1.86 | 0.03 | 21.2 | 0.35 |
| L2 | 3.11 | 0.31 | 1.75 | 0.03 | 20.8 | 0.50 |
| L3 | 7.14 | 2.40 | 1.88 | 0.03 | 21.3 | 0.74 |
| L4 | 2.08 | 0.19 | 1.72 | 0.08 | 21.1 | 1.11 |
| L5 | 1.92 | 0.29 | 1.73 | 0.03 | 21.2 | 0.76 |

As shown by Table 12 and FIG. 7, DNA from whole blood lysed with additives L1-L5 can be purified without protease incubation or adjustment of binding conditions. The purified DNA is of high purity, can be amplified in a real time TaqMan® assay and is visible as an intact band on an agarose gel.

The DNA is stable in additives L1-L5 for 28 days at 25° C.

### Example 7

Venous whole blood (1 ml) from one donor was drawn into a Vacutainer® blood collection tube (13 mm×75 mm) without anticoagulant. Directly after blood collection, the Vacutainer® tube was opened and 3 ml lysis buffer L1 was added. Blood and lysis buffer were mixed by inverting the tube 3 times. The lysates were stored at room temperature.

The composition of the additive is shown in Table 13:

TABLE 13

| Additive/ | |
|---|---|
| Buffer | Composition |
| L1 | 7 M urea, 2 M LiCl, 10% Triton ® X-100, 50 mM TRIS |
| | pH 8 |

Eight tubes with additive L1 were stored within the primary blood collection tubes for 9 days at room temperature. The lysates were refilled into empty primary blood collection tubes and processed along with the lysates from additives L1. Processing on BioRobot® MDx was performed with 600 μl lysate with additive L1 according to the procedure described in Example 3. The eluted DNA was analyzed as described in Example 1.

The results are shown in Table 14, with the gel shown in FIG. 8.

TABLE 14

| Addi- | AV yield | SD | AV | SD | PCR | SD |
|---|---|---|---|---|---|---|
| tive | [μg] | yield | A260/A280 | A260/A280 | AV CT | CT |
| L1 | 4.86 | 0.55 | 1.91 | 0.04 | 22.4 | 0.64 |

As shown by Table 14 and FIG. 8, DNA from additive L1 can be purified without incubations or adjustments. The purified DNA is of high purity, can be amplified in a real time TaqMan® assay and is visible as an intact band on an agarose gel.

The DNA is stable in the additive L1 for at least 9 days at room temperature

### Example 8

Venous whole blood (9 ml) from two donors were drawn into Monovette® blood collection devices (Sarstedt) without anticoagulant. Directly after collection, blood from the devices was mixed with additive L1 in a ratio of 1 volume blood and 3, 4, or 5 volumes additive.

The composition of the additive is shown in Table 15:

TABLE 15

| Additive | composition |
|---|---|
| L1 | 6.0 M sodium iodide, 12% Triton ® X-100, 50 mM TRIS |
| | pH 6 |

The lysate was stored for 2 days at room temperature.

For total DNA extraction on the BioRobot® MDx, 8 samples of 800 μl (ratio 1 plus 3) or 1000 μl (ratio 1 plus 4 and 1 plus 5) of lysate from each donor were transferred into wells of a 96 well QIAprep® plate (QIAGEN®). After the complete transfer of all samples, the lysates were sucked through the silica membrane by vacuum. Bound DNA was subsequently washed with 520 μl washing buffer 1,775 μl washing buffer 2, and 1020 μl ethanol. Each washing solution was applied with a dispenser and sucked through the membrane by vacuum. After drying, the total DNA was eluted from the membrane with 200 μl water.

The eluted DNA was analyzed as described in Example 1.

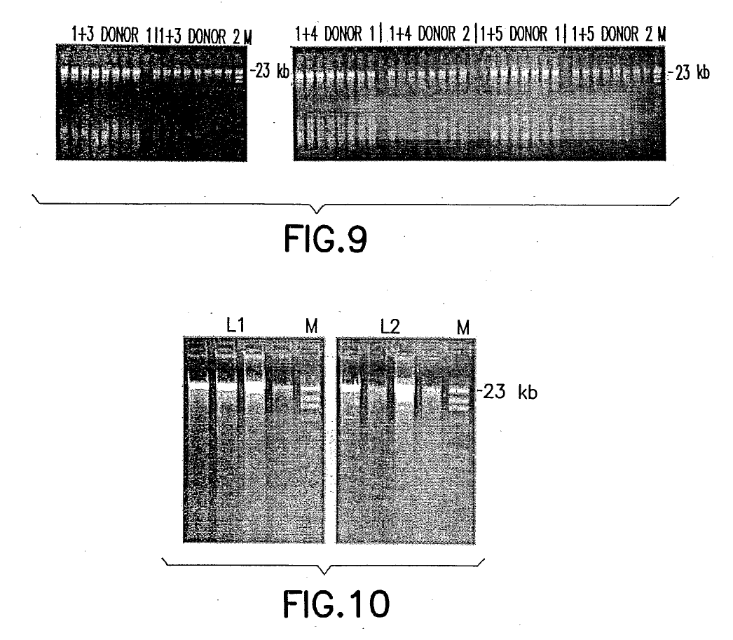

The results are shown in Table 16, with the gel shown in FIG. 9.

TABLE 16

| | | | | AV | SD | | |
|---|---|---|---|---|---|---|---|
| | | AV yield | SD | A260/ | A260/ | PCR | SD |
| Donor | Ratio | [μg] | yield | A280 | A280 | AV CT | CT |
| 1 | 1 + 3 | 5.0 | 0.4 | 1.82 | 0.02 | 22.28 | 0.53 |
| 2 | 1 + 3 | 3.2 | 0.2 | 1.72 | 0.03 | 23.86 | 0.48 |
| 1 | 1 + 4 | 4.3 | 0.4 | 1.84 | 0.03 | 22.23 | 0.38 |
| 2 | 1 + 4 | 3.1 | 0.1 | 1.76 | 0.03 | 23.56 | 0.68 |
| 1 | 1 + 5 | 4.2 | 0.2 | 1.83 | 0.04 | 22.37 | 0.49 |
| 2 | 1 + 5 | 2.5 | 0.2 | 1.74 | 0.02 | 22.90 | 0.81 |

As shown by Table 16 and FIG. 9, DNA from whole blood lysed with additive L1 can be purified without further incubations or adjustments on the automated platform BioRobot® MDx. The purified DNA is of high purity, can be amplified in a real time TaqMan® assay and is visible as an intact band on an agarose gel.

### Example 9

Saliva from four different donors was collected into 50 ml Falcon tubes (Becton, Dickinson and Company); 0.5 ml of saliva was added into an primary blood collection tubes (13 mm×75 mm, Becton, Dickinson and Company) filled with 2 ml additive L1 or L2. Saliva and additive were mixed by inverting the tube 3 times. After mixing, 40 μl of QIAGEN® protease were added into each sample, the lysates were mixed again and stored for 3 days at room temperature. Processing on BioRobot® MDx was performed with 1 ml lysate according to the procedure described in Example 3. The eluted DNA was analyzed as described in Example 1.

The composition of the additive is shown in Table 17:

TABLE 17

| Additive | Composition |
|---|---|
| L1 | 4.6 M guanidinium hydrochloride, 8.5% Triton, |
| | 50 mM TRIS ® X-100 pH 6 |
| L2 | 3.6 M guanidinium hydrochloride, 6.5% Triton, |
| | 50 mM TRIS ® X-100 pH 6 |

The results are shown in Table 18, with the gel shown in FIG. 10:

TABLE 18

| Addi- | AV yield | SD | AV | SD | PCR | SD |
|---|---|---|---|---|---|---|
| tive | [μg] | yield | A260/A280 | A260/A280 | AV CT | CT |
| L1 | 2.22 | 1.42 | 1.72 | 0.13 | 22.3 | 0.99 |
| L2 | 2.10 | 1.35 | 1.55 | 0.30 | 22.9 | 1.68 |

As shown by Table 18 and FIG. 10, DNA can be purified from saliva using additives L1 and L2 without adjustments of binding conditions. The purified DNA is of high purity, can be amplified in a real time TaqMan® assay and is visible as an intact band on an agarose gel.

### Example 10

Venous whole blood (9 ml) was drawn into Monovette® blood collection devices (Sarstedt) without anticoagulant. Directly after collection, blood from the devices was mixed with additive L1 in a ratio of 1 volume blood and 3.5, 4, or 5 volumes additive. Half of the samples contained MagAttract® Suspension B (magnetic particles, QIAGEN®, 300 μl per ml blood) as a solid nucleic acid binding phase.

The composition of the additive is shown in Table 19:

TABLE 19

| Additive | Composition |
|---|---|
| L1 | 5.7 M Guanidinium thiocyanate, 2% (w/v) Triton ® |
| | X-100, 19 mM Na₂-EDTA × 2H₂O, 47 mM NH₄Cl, |
| | 45 mM TRIS pH 8.5 |

The lysate was stored for 0, 7 and 14 days at room temperature.

For total DNA extraction, 450 μl (ratio 1 plus 3.5), 500 μl (ratio 1 plus 4), and 600 μl (ratio 1 plus 5) lysate were transferred into sample tubes on the BioRobot® M48 (QIAGEN). 30 μl MagAttract® Suspension B were added to those lysates that did not contain magnetic particles during storage. Bound DNA was subsequently washed with 1000 μl washing buffer 1, 1000 μl washing buffer 2, and 1000 μl H₂O. The DNA was eluted from the particles with 200 μl water.

The eluted DNA was analyzed as described in Example 1 except that the real-time PCR was not performed.

The results are shown in Table 20 and 21.

TABLE 20
storage with magnetic particles

| | | AV yield | SD | AV | SD |
|---|---|---|---|---|---|
| Storage | Ratio | [μg] | yield | A260/A280 | A260/A280 |
| 0 | 1 + 3.5 | 0.90 | 0.11 | 1.83 | 0.06 |
| 0 | 1 + 4 | 1.04 | 0.05 | 1.84 | 0.02 |
| 0 | 1 + 5 | 0.96 | 0.06 | 1.84 | 0.03 |
| 7 | 1 + 3.5 | 0.89 | 0.02 | 1.84 | 0.02 |
| 7 | 1 + 4 | 0.92 | 0.03 | 1.85 | 0.02 |
| 7 | 1 + 5 | 0.93 | 0.05 | 1.87 | 0.08 |
| 14 | 1 + 3.5 | 0.87 | 0.03 | 1.83 | 0.04 |
| 14 | 1 + 4 | 0.91 | 0.02 | 1.83 | 0.03 |
| 14 | 1 + 5 | 0.78 | 0.02 | 1.84 | 0.02 |

TABLE 21
storage without magnetic particles

| | | AV yield | SD | AV | SD |
|---|---|---|---|---|---|
| Storage | Ratio | [μg] | yield | A260/A280 | A260/A280 |
| 0 | 1 + 3.5 | 1.23 | 0.04 | 1.85 | 0.02 |
| 0 | 1 + 4 | 1.25 | 0.02 | 1.84 | 0.03 |
| 0 | 1 + 5 | 1.33 | 0.01 | 1.84 | 0.02 |
| 7 | 1 + 3.5 | 1.28 | 0.03 | 1.84 | 0.02 |
| 7 | 1 + 4 | 1.29 | 0.06 | 1.86 | 0.03 |
| 7 | 1 + 5 | 1.29 | 0.06 | 1.83 | 0.02 |
| 14 | 1 + 3.5 | 1.35 | 0.03 | 1.84 | 0.01 |
| 14 | 1 + 4 | 1.38 | 0.05 | 1.85 | 0.02 |
| 14 | 1 + 5 | 1.43 | 0.04 | 1.84 | 0.02 |

As can be drawn from Tables 20 and 21, DNA from whole blood lysed with additive L1 can be purified without further incubations or adjustments on the automated platform BioRobot M48. The purified DNA is of high purity. The solid nucleic acid binding phase may either be a component of the additive or added after storage.

The foregoing description of various aspects and embodiments of the invention exemplifies principles of the invention and does not limit the invention to the disclosed specific aspects and embodiments. A skilled artisan may make numerous variations of these embodiments without departing the spirit of the invention.

What is claimed:

1. A system comprising:
a container having an open top, a bottom, and a sidewall extending therebetween defining a chamber,
an additive disposed in the chamber, the additive comprising at least one chaotropic substance, at least one detergent, and at least one buffer.

2. The system of claim 1, wherein the container is a tube, the bottom is a closed bottom, the container further comprises a closure in the open top, and the chamber is at a reduced pressure.

3. The system of claim 2, wherein the closure is capable of being pierced with a needle or cannula, and wherein the reduced pressure is selected to draw a specified volume of a liquid sample into the chamber.

4. The system of claim 1, wherein the additive comprises an aqueous solution.

5. The system of claim 1, wherein the additive further comprises a chelating agent.

6. The system of claim 1, further comprising a solid phase disposed in the chamber, the solid phase capable of binding to nucleic acid.

7. The system of claim 1, wherein, upon disposition into the chamber of a biological sample comprising nucleic acid, the at least one chaotropic substance is present in an amount effective to denature proteins of cell membranes of cells in the biological sample thereby increasing permeability of the membranes, the at least one detergent is present in an amount effective to promote lysis of the cells or form complexes with the nucleic acid, and the at least one buffer is present in an amount effective to maintain a desired pH in the resulting sample/additive mixture.

8. The system of claim 1, wherein the at least one chaotropic substance is one or more compounds selected from the group consisting of lithium salts, sodium salts, urea, and guanidinium salts.

9. The system of claim 8, wherein the at least one chaotropic substance comprises one or more guanidinium salts selected from the group consisting of amino-substituted guanidinium salts and alkyl substituted guanidinium salts.

10. The system of claim 9, wherein the at least one chaotropic substance comprises one or more guanidinium salts selected from the group consisting of guanidinium hydrochloride and guanidinium thiocyanate.

11. The system of claim 10, wherein the at least one chaotropic substance comprises one or more guanidinium salts selected from the group consisting of amino-guanidinium hydrochloride, N,N′-diamino-guanidinium hydrochloride, and 1-methylguanidinium hydrochloride.

12. The system of claim 8, wherein the at least one chaotropic substance comprises one or more guanidinium salts present in an amount of 2 to 6 M.

13. The system of claim 1, wherein the at least one detergent comprises one or more detergents selected from the group consisting of polyoxyethylene sorbitan monolaurate, octylphenoxypolyethoxyethanol, and sodium dodecyl sulfate.

14. The system of claim 13, wherein the at least one detergent comprises octylphenoxypolyethoxyethanol.

15. The system of claim 1, wherein the at least one detergent is non-ionic.

16. The system of claim 13, wherein the at least one detergent is non-ionic.

17. The system of claim 1, wherein the at least one buffer comprises one or more buffers selected from the group consisting of TRIS, MOPS, MES, AND HEPES.

18. The system of claim 1, wherein the at least one buffer is present in an amount of 5 to 500 mM.

19. The system of claim 8, wherein the at least one chaotropic substance comprises a lithium salt present in an amount of 1 to 5 M.

20. The system of claim 8, wherein the at least one chaotropic substance comprises sodium iodide present in an amount of 4 to 6 M.

21. The system of claim 8, wherein the at least one chaotropic substance comprises urea present in an amount of 3 to 7 M.

22. The system of claim 8, wherein the at least one chaotropic substance comprises a guanidinium salt present in an amount of 2 to 6 M.

23. The system of claim 1, wherein the additive comprises 1 to 5 M lithium salt, 2.5 to 14% (v/v) Triton® X-100, and 10-100 mM TRIS.

24. The system of claim 23, wherein the TRIS has a pH of 6 to 8.

25. The system of claim 23, wherein the chamber is at a reduced pressure selected to draw a specified volume of a liquid sample into the chamber, and wherein the additive is disposed in the chamber such that the volumetric ratio of the additive to the specified volume of the liquid sample is 4:1 to 1:1.

26. The system of claim 25, wherein the sample is blood.

27. The system of claim 1, wherein the additive comprises 2-6 M guanidinium salt, 1.5-14% (v/v) Triton® X-100, and 10-100 mM TRIS

28. The system of claim 27, wherein the TRIS has a pH of 6 to 9.

29. The system of claim 27, wherein the chamber is at a reduced pressure selected to draw a specified volume of a liquid sample into the chamber, and wherein the additive is disposed in the chamber such that the volumetric ratio of the additive to the specified volume of the liquid sample is 4:1 to 1:1.

30. The system of claim 29, wherein the sample is blood.

31. The system of claim 30, wherein the guanidinium salt is selected from the group consisting of guanidinium hydrochloride, guanidinium thiocyanate, amino-guanidinium hydrochloride, N,N′-diamino-guanidinium hydrochloride and 1-methyl-guanidinium hydrochloride.

32. The system of claim 1, wherein the additive comprises 3 to 7 M urea, 5-14% (v/v) Triton® X-100, and 10 to 100 mM TRIS.

33. The system of claim 32, wherein the TRIS has a pH of 6 to 8.

34. The system of claim 32, wherein the chamber is at a reduced pressure selected to draw a specified volume of a liquid sample into the chamber, and wherein the additive is disposed in the chamber such that the volumetric ratio of the additive to the specified volume of the liquid sample is 6:1 to 2.5:1.

35. The system of claim 34, wherein the sample is blood.

36. The system of claim 1, wherein the additive comprises 3 to 7 M urea, 1.5 to 20 (v/v) SDS, and 10 to 100 mM TRIS.

37. The system of claim 36, wherein the TRIS has a pH of 6 to 8.

38. The system of claim 36, wherein the chamber is at a reduced pressure selected to draw a specified volume of a liquid sample into the chamber, and wherein the additive is disposed in the chamber such that the volumetric ratio of the additive to the specified volume of the liquid sample is 6:1 to 2.5:1.

39. The system of claim 38, wherein the sample is blood.

40. The system of claim 1, wherein the additive comprises 3 to 7 M urea, 1 to 2 M lithium chloride, 5-14% (v/v) Triton® X-100, and 10 to 100 mM TRIS.

41. The system of claim 40, wherein the TRIS has a pH of 6 to 8.

42. The system of claim 40, wherein the chamber is at a reduced pressure selected to draw a specified volume of a liquid sample into the chamber, and wherein the additive is disposed in the chamber such that the volumetric ratio of the additive to the specified volume of the liquid sample is 6:1 to 2:1.

43. The system of claim 42, wherein the sample is blood.

44. The system of claim 1, wherein the additive comprises 2 to 5 M guanidinium salt, 1 to 3 M lithium chloride, 5-14% (v/v) Triton® X-100, and 10 to 100 mM TRIS.

45. The system of claim 44, wherein the TRIS has a pH of 6 to 8.

46. The system of claim 44, wherein the chamber is held at a reduced pressure selected to draw a specified volume of a liquid sample into the chamber, and wherein the additive is disposed in the chamber such that the volumetric ratio of the additive to the specified volume of the liquid sample is 4:1 to 2:1.

47. The system of claim 46, wherein the sample is blood.

48. The system of claim 1, wherein the additive comprises 4 to 6 M sodium iodide, 5-14% (v/v) Triton® X-100, and 10 to 100 mM TRIS.

49. The system of claim 48, wherein the TRIS has a pH of 6 to 8.

50. The system of claim 48, wherein the chamber is held at a reduced pressure selected to draw a specified volume of a liquid sample into the chamber, and wherein the additive is disposed in the chamber such that the volumetric ratio of the additive to the specified volume of the liquid sample is 6:1 to 3:1.

51. The system of claim 50, wherein the sample is blood.

52. A method comprising the step of withdrawing blood from a patient directly into a chamber of a container, the chamber comprising an additive that comprises at least one chaotropic substance present in an amount effective to denature proteins of cell membranes of cells in the blood thereby increasing permeability of the membranes, at least one detergent present in an amount effective to promote lysis of the cells or form complexes with the nucleic acid, and at least one buffer present in an amount effective to maintain a desired pH in the resulting sample/additive mixture.

53. The method of claim 52 wherein the chamber is at a reduced pressure selected to draw a specified volume of a liquid sample into the chamber.

54. A method comprising the steps of:
disposing a biological sample comprising nucleic acid into a container comprising a chamber and an additive disposed in the chamber, wherein the additive comprises at least one chaotropic substance, at least one detergent, and at least one buffer;
transferring at least a portion of the disposed sample from the container onto a component capable of binding to nucleic acid, in the absence of a step of mixing the disposed sample or the transferred portion with reagents, buffers, or enzymes after the disposing step and prior to the transferring step;
washing the transferred sample; and
eluting nucleic acid from the component.

55. The method of claim 54, wherein, upon disposition into the chamber of the biological sample comprising nucleic acid, the at least one chaotropic substance denatures proteins of cell membranes of cells in the biological sample thereby increasing permeability of the membranes, the at least one detergent promotes lysis of the cells or form complexes with the nucleic acid, and the at least one buffer maintains a desired pH in the resulting sample/additive mixture.

56. The method of claim 54, wherein the at least one chaotropic substance is one or more compounds selected from the group consisting of lithium salts, sodium salts, urea, and guanidinium salts.

57. The method of claim 56, wherein the at least one chaotropic substance comprises one or more guanidinium salts selected from the group consisting of amino-substituted guanidinium salts and alkyl substituted guanidinium salts.

58. The method of claim 57, wherein the at least one chaotropic substance comprises one or more guanidinium salts selected from the group consisting of guanidinium hydrochloride and guanidinium thiocyanate.

59. The method of claim 58, wherein the at least one chaotropic substance comprises one or more guanidinium salts selected from the group consisting of amino-guanidinium hydrochloride, N,N′-diamino-guanidinium hydrochloride, and 1-methylguanidinium hydrochloride.

60. The method of claim 54, wherein the at least one chaotropic substance comprises one or more guanidinium salts present in an amount of 2 to 6 M.

61. The method of claim 54, wherein the at least one detergent comprises one or more detergents selected from the group consisting of polyoxyethylene sorbitan monolaurate, octylphenoxypolyethoxyethanol, and sodium dodecyl sulfate.

62. The method of claim 61, wherein the at least one detergent comprises octylphenoxypolyethoxyethanol.

63. The method of claim 54, wherein the at least one detergent is non-ionic.

64. The method of claim 61, wherein the at least one detergent is present in an amount of 1.5 to 20% (v/v).

65. The method of claim 54, wherein the at least one buffer comprises one or more buffers selected from the group consisting of TRIS, MOPS, MES, AND HEPES.

66. The method of claim 65, wherein the at least one buffer is present in an amount of 5 to 500 mM.

67. The method of claim 54, wherein the disposing step comprises drawing blood directly from a patient into the container.

68. The method of claim 67, wherein the container further comprises a closure in the open top, wherein the chamber is at a reduced pressure, and wherein the disposing step comprises piercing the closure with a needle or cannula such that a specified volume of the biological sample is drawn into the chamber.

69. The system of claim 54, wherein the additive comprises 1 to 5 M lithium salt, 2.5 to 14% (v/v) Triton® X-100, and 10-100 mM TRIS.

70. The system of claim 69, wherein the TRIS has a pH of 6 to 8.

71. The system of claim 54, wherein the additive comprises 2-6 M guanidinium salt, 1.5-14% (v/v) Triton® X-100, and 10-100 mM TRIS

72. The system of claim 71, wherein the TRIS has a pH of 6 to 9.

73. The system of claim 54, wherein the additive comprises 3 to 7 M urea, 5-14% (v/v) Triton® X-100, and 10 to 100 mM TRIS.

74. The system of claim 73, wherein the TRIS has a pH of 6 to 8.

75. The system of claim 54, wherein the additive comprises 3 to 7 M urea, 1.5 to 20 (v/v) SDS, and 10 to 100 mM TRIS.

76. The system of claim 75, wherein the TRIS has a pH of 6 to 8.

77. The system of claim 54, wherein the additive comprises 3 to 7 M urea, 1 to 2 M lithium chloride, 5-14% (v/v) Triton® X-100, and 10 to 100 mM TRIS.

78. The system of claim 77, wherein the TRIS has a pH of 6 to 8.

79. The system of claim 54, wherein the additive comprises 2 to 5 M guanidinium salt, 1 to 3 M lithium chloride, 5-14% (v/v) Triton® X-100, and 10 to 100 mM TRIS.

80. The system of claim 79, wherein the TRIS has a pH of 6 to 8.

81. The system of claim 54, wherein the additive comprises 4 to 6 M sodium iodide, 5-14% (v/v) Triton® X-100, and 10 to 100 mM TRIS.

82. The system of claim 81, wherein the TRIS has a pH of 6 to 8.

83. A method comprising the steps of:
disposing a biological sample comprising nucleic acid into a container comprising a chamber and an additive disposed in the chamber, wherein the additive comprises at least one chaotropic substance, at least one detergent, at least one buffer, and a solid phase capable of binding to nucleic acid;
transferring at least a portion of the disposed sample from the container into a second container, in the absence of a step of mixing the disposed sample or the transferred portion with reagents, buffers, or enzymes after the disposing step and prior to the transferring step;
washing the transferred sample; and
eluting nucleic acid from the component.

84. The method of claim 83, wherein, upon disposition into the chamber of the biological sample comprising nucleic acid, the at least one chaotropic substance denatures proteins of cell membranes of cells in the biological sample thereby increasing permeability of the membranes, the at least one detergent promotes lysis of the cells or form complexes with the nucleic acid, and the at least one buffer maintains a desired pH in the resulting sample/additive mixture.

85. The method of claim 83, wherein the at least one chaotropic substance is one or more compounds selected from the group consisting of lithium salts, sodium salts, urea, and guanidinium salts.

86. The method of claim 85, wherein the at least one chaotropic substance comprises one or more guanidinium salts selected from the group consisting of amino-substituted guanidinium salts and alkyl substituted guanidinium salts.

87. The method of claim 86, wherein the at least one chaotropic substance comprises one or more guanidinium salts selected from the group consisting of guanidinium hydrochloride and guanidinium thiocyanate.

88. The method of claim 87, wherein the at least one chaotropic substance comprises one or more guanidinium salts selected from the group consisting of amino-guanidinium hydrochloride, N,N′-diamino-guanidinium hydrochloride, and 1-methylguanidinium hydrochloride.

89. The method of claim 83, wherein the at least one chaotropic substance comprises one or more guanidinium salts present in an amount of 2 to 6 M.

90. The method of claim 83, wherein the at least one detergent comprises one or more detergents selected from the group consisting of polyoxyethylene sorbitan monolaurate, octylphenoxypolyethoxyethanol, and sodium dodecyl sulfate.

91. The method of claim 90, wherein the at least one detergent comprises octylphenoxypolyethoxyethanol.

92. The method of claim 83, wherein the at least one detergent is non-ionic.

93. The method of claim 90, wherein the at least one detergent is present in an amount of 1.5 to 20% (v/v).

94. The method of claim 83, wherein the at least one buffer comprises one or more buffers selected from the group consisting of TRIS, MOPS, MES, AND HEPES.

95. The method of claim 94, wherein the at least one buffer is present in an amount of 5 to 500 mM.

96. The method of claim 83, wherein the disposing step comprises drawing blood directly from a patient into the container.

97. The method of claim 96, wherein the container further comprises a closure in the open top, wherein the chamber is at a reduced pressure, and wherein the disposing step comprises piercing the closure with a needle or cannula such that a specified volume of the biological sample is drawn into the chamber.

98. The system of claim 83, wherein the additive comprises 1 to 5 M lithium salt, 2.5 to 14% (v/v) Triton® X-100, and 10-100 mM TRIS.

99. The system of claim 98, wherein the TRIS has a pH of 6 to 8.

100. The system of claim 83, wherein the additive comprises 2-6 M guanidinium salt, 1.5-14% (v/v) Triton® X-100, and 10-100 mM TRIS

101. The system of claim 100, wherein the TRIS has a pH of 6 to 9.

102. The system of claim 83, wherein the additive comprises 3 to 7 M urea, 5-14% (v/v) Triton® X-100, and 10 to 100 mM TRIS.

103. The system of claim 102, wherein the TRIS has a pH of 6 to 8.

104. The system of claim 83, wherein the additive comprises 3 to 7 M urea, 1.5 to 20 (v/v) SDS, and 10 to 100 mM TRIS.

105. The system of claim 104, wherein the TRIS has a pH of 6 to 8.

106. The system of claim 83, wherein the additive comprises 3 to 7 M urea, 1 to 2 M lithium chloride, 5-14% (v/v) Triton® X-100, and 10 to 100 mM TRIS.

107. The system of claim 106, wherein the TRIS has a pH of 6 to 8.

108. The system of claim 83, wherein the additive comprises 2 to 5 M guanidinium salt, 1 to 3 M lithium chloride, 5-14% (v/v) Triton® X-100, and 10 to 100 mM TRIS.

109. The system of claim 108, wherein the TRIS has a pH of 6 to 8.

110. The system of claim 83, wherein the additive comprises 4 to 6 M sodium iodide, 5-14% (v/v) Triton® X-100, and 10 to 100 mM TRIS.

111. The system of claim 110, wherein the TRIS has a pH of 6 to 8.
